(19) United States
(12) Patent Application Publication
(10) Pub. No.: US 2019/0030109 A1
(43) Pub. Date: Jan. 31, 2019

(54) A PHYTOCHEMICAL COMPOSITION FOR SICKLE CELL ANEMIA TREATMENT

(71) Applicants: Arun Prakash SHARMA, Bhind (IN); Anil Gyan BHANSALI, Bhind (IN)

(72) Inventors: Arun Prakash SHARMA, Bhind (IN); Anil Gyan BHANSALI, Bhind (IN)

(21) Appl. No.: 16/072,039

(22) PCT Filed: Jan. 23, 2016
(86) PCT No.: PCT/IB2016/050349

§ 371 Date: Jul. 23, 2018

(51) Int. Cl. A61K 36/61; A61P 7/06; A61K 36/9066; A61K 9/00; A61K 36/67; A61K 36/899; A61K 47/02

(52) U.S. Cl. CPC A61K 36/61; A61P 7/06; A61K 36/9066; A61K 47/02; A61K 36/67; A61K 36/899; A61K 9/0053

(57) ABSTRACT

A phytochemical composition for treatment of sickle cell anemia has been developed. The composition is a solid mixture of inorganic salts and various organic compounds of herbal origin and obtained by extracting a mixture of five herbs namely Piper guineenses seeds, Pterocarpus osum stem, Eugenia carophyllum fruit, Sorghum bicolor leaves and Curcuma longa tuber with aquous solution of sodium/potassium bicarbonate and sodium/potassium carbonate and then concentrating the extract to obtain a powder. Also described are methods of preparation of extraction product and method of its use for treatment of sickle cell disease afflicted patients.

### TECHNICAL FIELD

The present invention relates to the field of herbal drugs and in particular the invention relates to herbal drugs for treatment and management of sickle cell disease and methods of preparing and using it.

### BACKGROUND ART

Sickle-cell anemia (also known as sickle-cell disorder or sickle-cell disease) is a common genetic condition due to a haemoglobin disorder—inheritance of mutant haemoglobin genes from both parents. Such haemoglobinopathies, mainly thalassaemias and sickle-cell anemia, are globally widespread. About 5% of the world's population carries genes responsible for haemoglobinopathies. Each year about 300000 infants are born with major haemoglobin disorders—including more than 200000 cases of sickle-cell anemia in Africa. Globally, there are more carriers (i.e. healthy people who have inherited only one mutant gene from one parent) of thalassaemia than of sickle-cell anemia, but the high frequency of the sickle-cell gene in certain areas leads to a high rate of affected newborns.

Sickle-cell anemia is particularly common among people whose ancestors come from sub-Saharan Africa, India, Saudi Arabia and Mediterranean countries. Migration raised the frequency of the gene in the American continent. In some areas of sub-Saharan Africa, up to 2% of all children are born with the condition. In broad terms, the prevalence of the sickle-cell trait (healthy carriers who have inherited the mutant gene from only one parent) ranges between 10% and 40% across equatorial Africa and decreases to between 1% and 2% on the North African coast and <1% in South Africa. This distribution reflects the fact that sickle-cell trait confers a survival advantage against malaria and that selection pressure due to malaria has resulted in high frequencies of the mutant gene especially in areas of high malarial transmission. In West African countries such as Ghana and Nigeria, the frequency of the trait is 15% to 30% whereas in Uganda it shows marked tribal variations, reaching 45% among the Baamba tribe in the west of the country.

Frequencies of the carrier state determine the prevalence of sickle-cell anemia at birth. For example, in Nigeria, by far the most populous country in the subregion, 24% of the population are carriers of the mutant gene and the prevalence of sickle-cell anemia is about 20 per 1000 births. This means that in Nigeria alone, about 150 000 children are born annually with sickle-cell anemia.

The sickle-cell gene has become common in Africa because the sickle-cell trait confers some resistance to falciparum malaria during a critical period of early childhood, favoring survival of the host and subsequent transmission of the abnormal haemoglobin gene. Although a single abnormal gene may protect against malaria, inheritance of two abnormal genes leads to sickle-cell anemia and confers no such protection, and malaria is a major cause of ill-health and death in children with sickle-cell anemia. There is increasing evidence that malaria not only influences outcome but also changes the manifestations of sickle-cell anemia in Africa.

The public health implications of sickle-cell anemia are significant. Its impact on human health may be assessed against the yardsticks of infant and under-five mortality. As not all deaths occur in the first year of life, the most valid measure is under-five deaths. An increasing proportion of affected children now survive past five years of age but remain at risk of premature death. When health impact is measured by under-five mortality, sickle-cell anemia contributes the equivalent of 5% of under-five deaths on the African continent, more than 9% of such deaths in West Africa, and up to 16% of under-five deaths in individual West African countries.

In the United States of America median survival was estimated in 1994 to be 42 years for men and 48 years for women, whereas comparable figures for Jamaica published in 2001 suggested 53 years for men and 58.5 years for women. In Jamaica, the greatest mortality occurs between 6 and 12 months old when 10% of patients die despite considerable experience in the diagnosis and therapy of the condition and absence of malaria. There are, however, no firm data on the survival of patients with sickle-cell anemia on the African continent. In sub-Saharan Africa mortality will be much higher than in Jamaica, and in some areas estimates derived from the age structure of populations attending clinics suggest that half of those with sickle-cell anemia have died by the age of five years usually from infections including malaria and pneumococcal sepsis, and from the anemia itself.

Sickle-cell anemia covers a wide spectrum of illness. Most affected people have chronic anemia with a haemoglobin concentration of around 8 g/dl. The main problems arise from the tendency of the red blood cells to become sickle-shaped and block capillaries at low oxygen tension. In children, sickle-shaped red blood cells often become trapped in the spleen, leading to a serious risk of death before the age of seven years from a sudden profound anemia associated with rapid splenic enlargement or because lack of splenic function permits an overwhelming infection. Between 6 and 18 months of age affected children most often present with painful swelling of the hands and/or feet (hand-foot syndrome). Survivors may also suffer recurrent and unpredictable severe painful crises, as well as “acute chest syndrome” (pneumonia or pulmonary infarction), bone or joint necrosis, priapism or renal failure. For most patients the incidence of complications can be reduced by simple protective measures such as prophylactic administration of penicillin in childhood, avoiding excessive heat or cold and dehydration, and contact as early as possible with a specialist centre. These precautions are most effective if susceptible infants are identified at birth. Some patients have such severe problems that they need regular blood transfusion and iron-chelation therapy.

Over the past 10 years, progress has been made in several respects: long-term treatment with hydroxyurea has decreased the rate of painful crises and improved the quality of life of patients with sickle-cell anemia; imaging studies can help in the prompt management of life-threatening complications, such as stroke and the chest syndrome; bone marrow transplantation, although not free of risk and not available for all patients, can cure sickle-cell anemia; regular blood-transfusion programmes associated with iron chelation can prevent complications; gene therapy has been carried out successfully in animal models, but has yet to be tested in clinical trials in human subjects. Consequently, it is possible to provide a better quality of life, and, in some cases, a definitive cure for patients with sickle-cell anemia. However, these advances, which are mainly applicable in high-resource countries, have unfortunately widened the gap in terms of quality of life between patients in developed countries and those in developing countries, and that gap can be reduced only through a general improvement in health services.

Nprisan (U.S. Pat. No. 5,800,819) is a herbal drug available for treatment of Sickle-cell anemia. It was developed by NIPRD Nigeria. Since it has been found to be only ˜75% effective in clinical trials, Therefore, there is an urgent need to develop an alternative low cost safe drug for treatment of Sickle-cell anemia having efficacy >95%.

### DISCLOSURE OF THE INVENTION

### Technical Problem

Currently, there is no effective and safe drug available for treatment of Sickle-cell anemia.

It is a primary objective of the present invention to provide a safe and cost effective drug that can provide relief to Sickle-cell anemia patients from various complications arising due to this anemia.

Additionally, it is also an objective of this invention to provide methods of preparation of drug and methods of treatment of sickle cell anemia patients with the drug.

### Technical Solution

The present invention provides a composition for treating Sickle-cell anemia comprising inorganic salts such as sodium/potassium bicarbonate and sodium/potassium carbonate and various organic compounds obtained by extracting a mixture of five herbs namely Piper guineenses seeds, Pterocarpus osum stem, Eugenia carophyllum fruit, Sorghum bicolor leaves and Curcuma longa tubers. In its preferred form of the invention, the mixture may include from about 5% to 15% by weight of said Piper guineenses seeds, 20%-30% by weight of said Pterocarpus osum stem, 5% to 15% of said Eugenia carophyllum fruit, 10%-20% of said Sorghum bicolor leaves and 5% to 15% of said Curcuma longa tuber and 20% to 40% of said salts. In its presently most preferred form, the mixture may include about 7.5% by weight of said Piper guineenses seeds, 28% by weight of said Pterocarpus osum stem, 7.5% of said Eugenia carophyllum fruit, 14% of said Sorghum bicolor leaves and 10% of said Curcuma longa tuber and 33% of said salts.

This invention also provides method of preparation of a composition for treatment of Sickle-cell anemia The method comprises mixing of milled Piper guineenses seeds, Pterocarpus osum stem, Eugenia carophyllum fruit, Sorghum bicolor leaves and Curcuma longa tuber, extraction from a aqueous solution of sodium/potassium bicarbonate and sodium/potassium carbonate, evaporation of aqueous solution to obtain a powder and then capsulation, tableting or syrup formation of the powder as such or with suitable excepient for administration to patients. A dose of 300 g to 500 mg is administered orally to Sickle-cell anemia patients.

In the present invention, there provides a composition for treating Sickle-cell anemia comprising inorganic salts and various organic compounds obtained by extracting a mixture of five herbs namely Piper guineenses seeds, Pterocarpus osum stem, Eugenia carophyllum fruit, Sorghum bicolor leaves and Curcuma longa tubers. In its preferred form of the invention, the mixture may include from about 5% to 15% by weight of said Piper guineenses seeds, 20%-30% by weight of said Pterocarpus osum stem, 5% to 15% of said Eugenia carophyllum fruit, 10%-20% of said Sorghum bicolor leaves and 5% to 15% of said Curcuma longa tuber and 20% to 40% of said sodium/potassium bicarbonate and sodium/potassium carbonate salts. In its presently most preferred form, the mixture may include about 7.5% by weight of said Piper guineenses seeds, 28% by weight of said Pterocarpus osum stem, 7.5% of said Eugenia carophyllum fruit, 14% of said Sorghum bicolor leaves and 10% of said Curcuma longa tuber and 33% of said sodium bicarbonate and sodium carbonate salt.

All the plant raw materials used in preparation of extracts are expected to contribute various terpenoids, flavonoids, alkaloids, tannins, glycoside and polyphenols in the extracts. These plant raw materials are known to have various medicinal properties. Piper guineenses seeds are known to contain tannins and flavonoids which are helpful in treatment of enteritis, gastritis and esophagitis, healing of wounds and inflammation of mucous membrane and have countless health benefits especially due to their antiviral, anti-tumor, anti-allergic anti-oxidant and anti-inflammatory abilities. Pterocarpus ossum stems are known for antimicrobial properties. Eugenia carophyllum fruits offer many health benefits such as helpful in digestion, having antimicrobial properties, fighting against cancer, protecting the liver, boosting the immune system, controlling diabetes, preserving bone quality, and containing anti-mutagenic properties.

Sorghum bicolor leaves are known to contain antioxidant compounds which protect against development of various types of cancers. These leaves also have tannins imparting anti-diabetic properties. They also contain compounds which boosts blood circulation and energy levels in body. Lastly Curcuma longa tubers have a wide range of biological effects. Prominant ones are immunity enhancement, anti inflammatory, antioxidant, antibacterial and antiviral activities. Considering various aforementioned biological properties, the extracts of these plant raw materials are expected to provide not only an effective medicine for treatment of sickle-cell anemia but also to boost their immunity mainly contributed by Curcuma longa.

The extract is prepared by dissolving Aq sodium bicarbonate and carbonate solution in water, adding all milled herbs, mixing them for a few hours and isolation of extract by filtration. The filtrate is concentrated till a powder is obtained. The milled powder is then formulated in capsules having 300 mg and 400 mg of powder.

### Advantageous Effects

In vitro tests carried out for demonstrating anti-sickling effect on the red blood cells of SCA patients, it was observed that the extract protected the RBC from sickling when these were exposed to low oxygen environment. The effect lasted for over 48 h with more than 91% protection. In already sickled red blood cells samples, dose dependent reversal lasting over 48 h was also observed with the extracts. Additionally, laboratory data clearly indicated the dose dependent reversal of already sickled red blood cells using the above described plant extracts. The reversal lasted for 48 h and the effect was 100%. These observation clearly demonstrates scientific justification of beneficial effects of the extracts. Safety studies conducted on extract do not show any toxicity and other adverse effects.

The result of clinical trials in human patients as volunteers demonstrated that the drug has no detectable adverse effects on kidney, liver and blood chemistry. On average, 90% of patients involved in the clinical trials have not experienced any major sickle cell disease crisis since they entered the clinical trial program. Even the few patients (10%) who have experienced the crisis reported that the such crisis were less frequent and less severe than what they experienced before entering the clinical trial program. The patients taking the drugs had improved weight and appetite. There was less incidence of anemia and jaundice. The positive sickling test of red blood cells with sodium metabisulfite shown by patients in the beginning of entering into clinical trials completely turned negative after about 8 months. This shows high efficacy of invented drugs.

### MODE FOR CARRYING OUT THE INVENTION

Preferred mode of the invention is illustrated by following examples:
1. Preparation of Herbal Extract: The extract is prepared by dissolving Aq sodium bicarbonate (8 Kg) and sodium carbonate (4 Kg) solution in potable water (400 L), adding all milled herbs (Piper 3 Kg, Sorghum 5.5 Kg, Eugenia 3 Kg, Petrocarpus 10 Kg, Curcuma 3.7 Kg), mixing them for a few hours and isolation of extract by filtration. The filtrate is concentrated by distillation below 50-60 C under vacuum to yield 5-6 Kg of a powder. 2. Preparation of Formulation: The powder obtained in example one is milled and filled in capsules as such or after mixing with common excepients. Then, it is filled in capsules to have dose of 300/400/500 mg. 3. Administration to Patients: The capsules are taken twice daily. The lower doses are recommended for children and the higher doses for adults.

1. A composition for treating sickle cell disease comprising a water extraction product of a mixture containing about 5% to 15% by weight of Piper guineenses seeds, 20%-30% by weight of Pterocarpus osum stem, 5% to 15% of said Eugenia carophyllum fruit, 10%-20% of Sorghum bicolor leaves and 5% to 15% of Curcuma longa tuber and 20% to 40% of sodium or potassium bicarbonate and sodium or potassium carbonate salts.

2. A composition as set forth in claim 1, wherein said mixture comprises about 7.5% by weight of said Piper guineenses seeds, 28% by weight of said Pterocarpus osum stem, 7.5% of said Eugenia carophyllum fruit, 14% of said Sorghum bicolor leaves and 10% of said Curcuma longa tuber and 33% of said sodium bicarbonate and sodium carbonate salt.

3. A composition for preparing a drug effective for treating sickle cell disease comprising a mixture containing about 5% to 15% by weight of Piper guineenses seeds, 20%-30% by weight of Pterocarpus osum stem, 5% to 15% of said Eugenia carophyllum fruit, 10%-20% of Sorghum bicolor leaves and 5% to 15% of Curcuma longa tuber and 20% to 40% of sodium or potassium bicarbonate and sodium or potassium carbonate salts.

4. A composition as set forth in claim 3, wherein said mixture comprises 7.5% by weight of said Piper guineenses seeds, 28% by weight of said Pterocarpus osum stem, 7.5% of said Eugenia carophyllum fruit, 14% of said Sorghum bicolor leaves and 10% of said Curcuma longa tuber and 33% of said sodium bicarbonate and sodium carbonate salt.

5. A method for preparing a composition for treating sickle cell anemia comprising following steps:
a. Weighing from about 5% to 15% by weight of Piper guineenses seeds, 20%-30% by weight of Pterocarpus osum stem, 5% to 15% of said Eugenia carophyllum fruit, 10%-20% of Sorghum bicolor leaves and 5% to 15% of Curcuma longa tuber and 20% to 40% of sodium or potassium bicarbonate and sodium or potassium carbonate salts;
b. Preparing a solution of alkali salts in water;
c. Extracting a mixture of said five herbs with said aquous solution of step b;
d. Converting of liquid extract into powder form by concentration.

6. A method as set forth in claim 5. Wherein said mixture comprises 7.5% by weight of said Piper guineenses seeds, 28% by weight of said Pterocarpus osum stem, 7.5% of said Eugenia carophyllum fruit, 14% of said Sorghum bicolor leaves and 10% of said Curcuma longa tuber and 33% of said sodium bicarbonate and sodium carbonate salt.

7. A method for treating a human afflicted with Sickle-cell anemia comprising:
a. Weighing from about 5% to 15% by weight of Piper guineenses seeds, 20%-30% by weight of Pterocarpus osum stem, 5% to 15% of said Eugenia carophyllum fruit, 10%-20% of Sorghum bicolor leaves and 5% to 15% of Curcuma longa tuber and 20% to 40% of sodium or potassium bicarbonate and sodium or potassium carbonate salts;
b. Preparing a solution of alkali salts in water;
c. Extracting a mixture of said five herbs with said aquous solution of step b;
d. Converting of liquid extract into powder form by concentration.
e. Preparing capsules/tablets or syrup by mixing with excepients
f. Treating a human afflicted with sickle cell anemia with formulation of said drug material.

8. A method as set forth in claim 7. Wherein said mixture comprises 7.5% by weight of said Piper guineenses seeds, 28% by weight of said Pterocarpus osum stem, 7.5% of said Eugenia carophyllum fruit, 14% of said Sorghum bicolor leaves and 10% of said Curcuma longa tuber and 33% of said sodium bicarbonate and sodium carbonate salt

9. A method as set forth in claim 8 where a dose of approximately 300-500 mg of said drug material is administered orally to said person afflicted with Sickle-cell anemia
